(19) United States
(12) Patent Application Publication
(10) Pub. No.: US 2025/0380895 A1
(43) Pub. Date: Dec. 18, 2025

(54) RECOVERY LEVEL ESTIMATION DEVICE, RECOVERY LEVEL ESTIMATION METHOD, AND RECORDING MEDIUM

(71) Applicant: NEC Corporation, Minato-ku, Tokyo (JP)

(72) Inventors: Toshinori HOSOI, Tokyo (JP); Shoji YACHDA, Tokyo (JP)

(73) Assignee: NEC Corporation, Minato-ku, Tokyo (JP)

(21) Appl. No.: 18/574,820

(22) PCT Filed: Jul. 6, 2021
(86) PCT No.: PCT/JP2021/025428

§ 371 Date: Jan. 24, 2025

(51) Int. Cl. A61B 5/00; A61B 3/113; A61B 3/14; A61B 5/16; G16H 50/70

(52) U.S. Cl. CPC A61B 5/4064; A61B 3/113; A61B 3/14; A61B 5/163; A61B 5/7275; A61B 5/7278; A61B 5/746; G16H 50/70

(57) ABSTRACT

In a recovery level estimation device, an image acquisition means acquires images capturing eyes of a target patient for whom a recovery level is estimated. An eye movement feature extraction means extracts an eye movement feature which is a feature of an eye movement of the target patient, based on the images. A past case storage means stores, as past cases, a plurality of cases in which each of patients and information concerning the eye movement feature and the recovery level of the patient are associated with each other. A similar case search means configured to search for each similar case including a similar eye movement feature to the eye movement feature of the target patient among the past cases. A recovery level estimation means estimates the recovery level of the target patient based on information concerning the recovery level of each similar case.

### TECHNICAL FIELD

The present disclosure relates to a technique for estimating a level of recovery of a patient.

### BACKGROUND ART

While healthcare costs are putting pressure on national finances worldwide, the number of patients with cerebrovascular diseases in Japan stands at 1,115,000, with annual healthcare costs amounting to 1.8 trillion yen or more. The number of stroke patients is expected to increase as the birthrate declines and the population ages; however, medical resources are limited, and there is a strong need for operational efficiency not only in acute care hospitals but also in convalescent rehabilitation hospitals.

Because cerebral infarction can cause a serious sequela unless emergency transport and measures are taken promptly after onset, it is important to detect and take measures as early as possible while symptoms are mild. Approximately half of the patients with cerebral infarction will develop cerebral infarction again within years and will likely recur the same type of cerebral infarction as the first. Therefore, there is also a strong need for early detection of signs of recurrence.

However, in order to measure a recovery level of a patient in a convalescent rehabilitation hospital, it is necessary for medical personnel to accompany the patient and conduct various tests, which are time-consuming and labor-intensive. Accordingly, the frequency of measuring a recovery level is reduced, feedback to patients and providers will be lost, and patients will be less motivated to rehabilitate, resulting in reduced rehabilitation volume and delayed review of inappropriate rehabilitation plans, which will reduce the effectiveness of recovery. In addition, signs of recurrence are difficult for the patient to recognize on his or her own and often do not occur in time for periodic examinations and medical examinations.

Patent document 1 describes a more objective quantification of recovery status related to gait, based on a movement of a patient and eye movements while walking. Patent document 2 describes the estimation of psychological states from features based on eye movements. Patent document 3 describes determining reflexivity of the eye movements under predetermined conditions. Patent document 4 describes estimating a recovery transition based on movement information quantified from data of a rehabilitation subject.

### PRECEDING TECHNICAL REFERENCES

### Patent Document

Patent Document 1: Japanese Laid-open Patent Publication No. 2019-067177

Patent Document 2: Japanese Laid-open Patent Publication No. 2017-202047

### SUMMARY

### Problem to be Solved by the Invention

Conventionally, estimation of a recovery level of a patient has been conducted by quantifying a recovery status by having medical personnel or a specialist visually or palpatively evaluate the patient performing a given operation. It is also known to quantify a recovery status of the patient in a remote location by transmitting a video of movements of the patient and a human body posture analysis result as data, and allowing the medical personnel or the specialist to visually evaluate the data. In addition, Patent Document 1 describes a medical information processing system which quantifies a recovery status by analyzing a manner in which a human body moves based on a video of a walking scene of the patient.

In order to estimate the recovery level using a traditional method, the patient needs to go to a hospital where the medical personnel and the specialist are available. However, many patients have difficulty going to the hospital for a variety of reasons. By transmitting patient data, hospital visits of the patient are reduced, but it requires a lot of time and effort on the medical personnel and other professionals to visually evaluate the patient data. Moreover, a method of quantifying recovery status based on the video of the walking scene does not require much effort on the medical personnel and the like, but it can only evaluate the patient who has recovered to a level where the patient can walk, and there is also the problem of a risk of falling when walking.

It is one object of the present disclosure to quantitatively estimate the recovery level without burdening the patient or the medical personnel.

### Means for Solving the Problem

According to an example aspect of the present disclosure, there is provided a recovery level estimation device including:
an image acquisition means configured to acquire images capturing eyes of a target patient for whom a recovery level is estimated;
an eye movement feature extraction means configured to extract an eye movement feature which is a feature of an eye movement of the target patient, based on the images;
a past case storage means configured to store, as past cases, a plurality of cases in which each of patients and information concerning the eye movement feature and the recovery level of the patient are associated with each other;
a similar case search means configured to search for each similar case including a similar eye movement feature to the eye movement feature of the target patient among the past cases; and
a recovery level estimation means configured to estimate the recovery level of the target patient based on information concerning the recovery level of each similar case.

According to another example aspect of the present disclosure, there is provided a recovery level estimation method including:
acquiring images capturing eyes of a target patient for whom a recovery level is estimated;
extracting an eye movement feature which is a feature of an eye movement of the target patient, based on the images;
searching for each similar case including a similar eye movement feature to the eye movement feature of the target patient among a plurality of past cases in which each of patients and information concerning the eye movement feature and the recovery level of the patient are associated with each other; and
estimating the recovery level of the target patient based on information concerning the recovery level of each similar case.

According to a further example aspect of the present disclosure, there is provided a recording medium storing a program, the program causing a computer to perform a process including:
acquiring images capturing eyes of a target patient for whom a recovery level is estimated;
extracting an eye movement feature which is a feature of an eye movement of the target patient, based on the images;
searching for each similar case including a similar eye movement feature to the eye movement feature of the target patient among a plurality of past cases in which each of patients and information concerning the eye movement feature and the recovery level of the patient are associated with each other; and
estimating the recovery level of the target patient based on information concerning the recovery level of each similar case.

### Effect of the Invention

According to the present disclosure, it becomes possible to quantitatively estimate a recovery level without burdening a patient or medical personnel.

### BRIEF DESCRIPTION OF THE DRAWINGS

FIG. 1 illustrates a schematic configuration of a recovery level estimation device.

FIG. 2 illustrates a hardware configuration of the recovery level estimation device.

FIG. 3 illustrates a functional configuration of the recovery level estimation device according to a first example embodiment.

FIG. 4A to FIG. D are examples of eye movement features.

FIG. 5 is a flowchart of a recovery level estimation process according to the first example embodiment.

FIG. 6 illustrates a functional configuration of a recovery level estimation device according to a second example embodiment.

FIG. 7 illustrates a flowchart of the recovery level estimation device according to the second example embodiment.

FIG. 8 illustrates a functional configuration of a recovery level estimation device according to a third example embodiment.

FIG. 9 illustrates a specific example of a task.

FIG. 10 illustrates a flowchart of a recovery level estimation process of the third example embodiment.

FIG. 11 illustrates a functional configuration of a recovery level estimation device according to a fourth example embodiment.

FIG. 12 illustrates a flowchart of a recovery level estimation process of the fourth example embodiment.

### EXAMPLE EMBODIMENTS

In the following, example embodiments will be described with reference to the accompanying drawings.

### First Example Embodiment

### Configuration

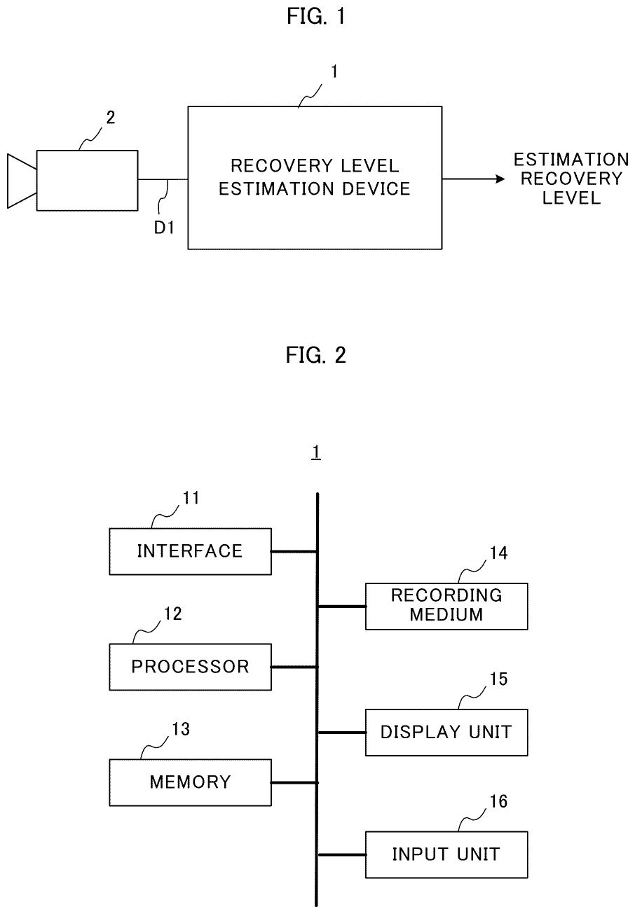

FIG. 1 illustrates a schematic configuration of a recovery level estimation device according to a first example embodiment of the present disclosure. A recovery level estimation device 1 is connected to a camera 2. The camera 2 captures eyes of a patient for whom a recovery level is estimated (hereinafter, simply referred to as a “target patient”), and transmits captured images D1 to the recovery level estimation device 1. The camera 2 is assumed to use a high-speed camera capable of capturing images of eyes at a high speed, for instance, 1,000 frames per second. The recovery level estimation device 1 estimates the recovery level by analyzing the captured images D1 and calculating an estimation recovery level.

FIG. 2 is a block diagram illustrating a hardware configuration of the recovery level estimation device 1. As illustrated, the recovery level estimation device 1 includes an interface (interface) 11, a processor 12, a memory 13, a recording medium 14, a display unit 15, and an input unit 16.

The interface 11 exchanges data with the camera 2. The interface 11 is used when receiving the captured images D1 generated by the camera 2. Moreover, the interface 11 is used when the recovery level estimation device 1 transmits and receives data to and from a predetermined device connected by a wired or wireless communication.

The processor 12 corresponds to one or more processors each being a computer such as a CPU (Central Processing Unit) and controls the whole of the recovery level estimation device 1 by executing programs prepared in advance. The memory 13 is formed by a ROM (Read Only Memory) and a RAM (Random Access Memory). The memory 13 stores the programs executed by the processor 12. Moreover, the memory 13 is used as a working memory during executions of various processes performed by the processor 12.

The recording medium 14 is a non-volatile and non-transitory recording medium such as a disk-shaped recording medium or a semiconductor memory and is formed to be detachable with respect to the recovery level estimation device 1. The recording medium 14 records the various programs executed by the processor 12. When the recovery level estimation device 1 executes a recovery level estimation process, a program recorded in the recording medium 14 is loaded into the memory 13 and executed by the processor 12.

The display unit 15 is, for instance, an LCD (Liquid Crystal Display and displays the estimation recovery level or the like which indicates a result of estimating the recovery level of the target patient. The display unit 15 may display the task of a third example embodiment to be described later. The input unit 16 is a keyboard, a mouse, a touch panel, or the like, and is used by an operator such as medical personnel or a specialist.

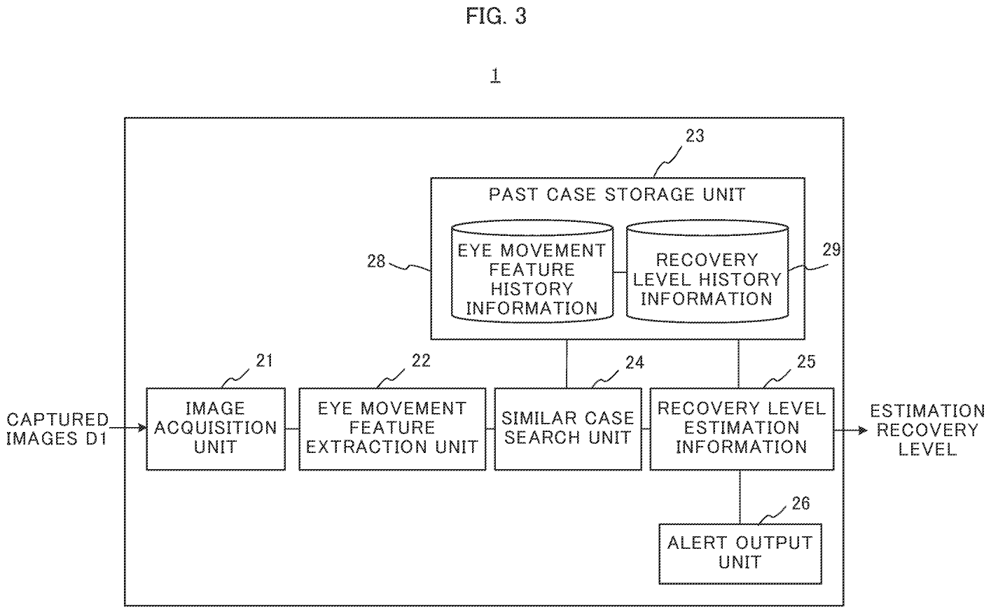

FIG. 3 is a block diagram illustrating a functional configuration of the recovery level estimation device 1. Functionally, the recovery level estimation device 1 includes an image acquisition unit 21, an eye movement feature extraction unit 22, a past case storage unit 23, a similar case search unit 24, a recovery level estimation unit 25, and an alert output unit 26. Note that the image acquisition unit 21, the eye movement feature extraction unit 22, the similar case search unit 24, the recovery level estimation unit 25, and the alert output unit 26 are realized by the processor 12 executing respective programs. Moreover, the past case storage unit 23 is realized by the memory 13.

The recovery level estimation device 1 estimates the recovery level by calculating an estimation recovery level for the target patient based on a past case and eye movement features of the target patient. Specifically, the recovery level estimation device 1, for instance, can be applied to the estimation of the recovery level by rehabilitation from a sequela due to a cerebral infarction.

The image acquisition unit 21 acquires the captured images D1 which are obtained by imaging the eyes of the target patient and supplied from the camera 2. Note that when the captured images D1 captured by the camera 2 are collected and stored in a database or the like, the image acquisition unit 21 may acquire the captured images D1 from the database or the like.

The eye movement feature extraction unit 22 performs a predetermined image process with respect to the captured images D1 acquired by the image acquisition unit 21, and extracts the eye movement feature of the target patient. In detail, the eye movement feature extraction unit 22 extracts time series information of a vibration pattern of the eyes in the captured images D1 as the eye movement feature.

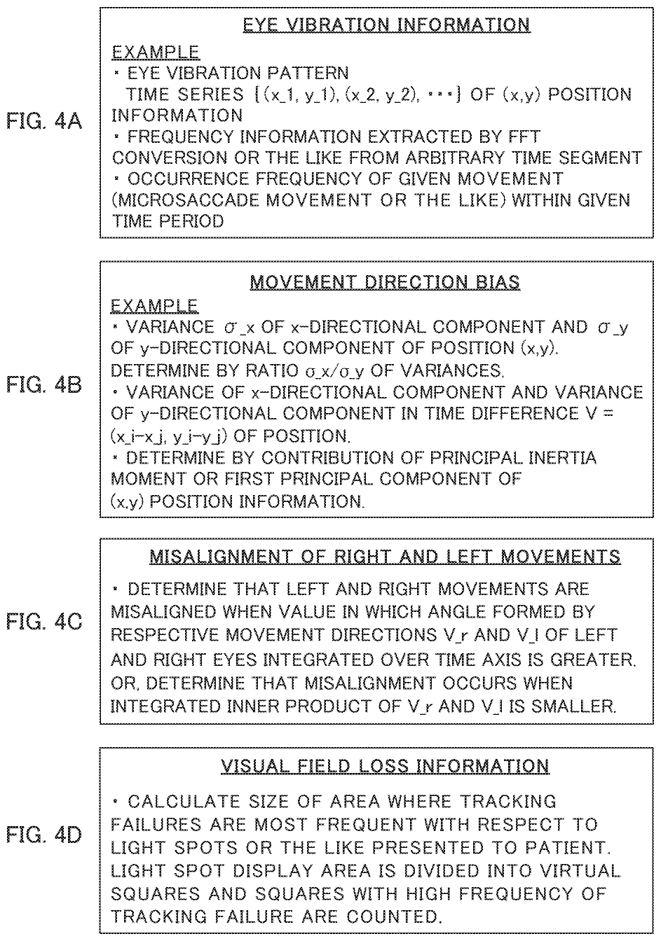

FIG. 4A to FIG. 4D illustrate examples of the eye movement features. Each eye movement feature is regarded as a feature of a human eye movement, for instance, eye vibration information, a bias in a movement direction, a misalignment of right and left movements, visual field defect information, or the like.

As illustrated in FIG. 4A, the eye vibration information is information concerning a vibration of the eyes. Based on the eye vibration information, for instance, abnormalities such as eye tremor and the like caused by the cerebral infarction can be detected. In detail, the eye vibration information may be, for each of a right eye and a left eye, information concerning a time-series change of the coordinates in which, for instance, xy coordinates of the center of a pupil may be taken, or may be frequency information extracted by a FFT (Fast Fourier Transform) or the like within any time segment. Alternatively, the eye vibration information may be information concerning an occurrence frequency within a given time of a predetermined movement such as microsaccard.

As illustrated in FIG. 4B, the bias in the movement direction is regarded as information concerning a bias of the movement between the eyes in a vertical direction or a lateral direction. Based on the bias of the movement direction, for instance, it is possible to detect an abnormality such as gaze paralysis or the like caused by the cerebral infarction. In detail, a variance of an x-directional component and a variance of a y-directional component of a position (x, y) are calculated and a ratio of the variances is used to determine the abnormality, or the variance of the x-directional component and the variance of the y-directional component are calculated regarding a time difference of a position of velocity information and a ratio of the variances is used to determine the abnormality, thereby obtaining information concerning a quantitative bias of the movement direction. Moreover, the bias of the movement direction may be determined and acquired based on a contribution ratio of a principal inertia moment or the first principal component of (x,y) position information.

As illustrated in FIG. 4C, the misalignment of the right and left movements is regarded as information concerning a misalignment of eye movements of the right and left eyes. Based on the misalignment of the right and left movements, for instance, it is possible to detect the abnormality such as strabismus or the like caused by the cerebral infarction. In detail, in a case where an angle between the movement directions of respective right and left eyes is totaled on a time axis, it is determined that the greater the totaled value, the greater the misalignment, or in a case where an inner product of angles formed by respective movement directions of the right and left eyes is totaled on the time axis, it is determined that the smaller the value obtained by totaling the inner products, the greater the misalignment, thereby it is possible to obtain information concerning a quantitative misalignment of the right and left movements.

As illustrated in FIG. 4D, the visual field defect information is information concerning a defect of a visual field. Based on the visual field defect information, for instance, it is possible to detect the abnormality such as gaze failure caused by the cerebral infarction. In detail, the target patient is asked to track a light spot being presented and a size of an area where a tracking failure occurs frequently is calculated, or a light spot display area is divided into virtual squares and squares with high frequency of the tracking failure are counted, thereby quantitative visual field loss information can be obtained.

The past case storage unit 23 stores eye movement feature history information 28 and recovery level history information 29 in which information concerning the eye movement features and the recovery level of each patient are associated with the patient with a target disease. The eye movement feature history information 28 records the eye movement features, measurement date, and the like of the patient in association with the patient identification information which identifies the patient. Also, the recovery level history information 29 stores the recovery level, the measurement date, and the like of the patient in association with the patient identification information. For the recovery level, for instance, a BBS (Berg Balance Scale), a TUG (Timed Up and Go test), a FIM (Functional Independence Measure), or the like can be arbitrarily applied. Thus, the past case storage unit 23 stores a plurality of cases in which the patient and information concerning the eye movement features and the recovery level of the patient are associated with each other as past cases.

The similar case search unit 24 searches, among the past cases, for a case including a similar eye movement feature to the eye movement feature of the target patient (hereinafter, referred to as a “similar case”). Specifically, the similar case search unit 24 retrieves and acquires, from the past case storage unit 23, the similar eye movement feature to the eye movement feature of the target patient, and the information concerning the patient identification information and the recovery level corresponding to the similar eye movement feature, as the similar case. The similar case search unit 24 may, for instance, retrieve similar cases for a predetermined number of cases in descending order of degrees of similarity, or may retrieve only one case having the highest degree of similarity.

The recovery level estimation unit 25 calculates the estimation recovery level of the target patient based on the information concerning the recovery level of the similar case. For instance, in a case where only one similar case having the highest degree of similarity from the past cases is retrieved, the recovery level estimation unit 25 determines the recovery level of the similar case as the estimation recovery level of the target patient. In a case where a predetermined number of the similar cases are retrieved from the past cases, the recovery level estimation unit 25 may use the most frequent recovery level as the estimation recovery level for the target patient, or may calculate an average value of the recovery levels to be the estimation recovery level for the target patient.

The alert output unit 26 refers to the memory 13, and outputs an alert to the target patient on the display unit 15 when the estimation recovery level of the target patient deteriorates below a threshold value. In a case where a time period is set for the alert and the estimation recovery level of the target patient deteriorates below the threshold value within a predetermined time period, the alert is output.

### Recovery Estimation Process

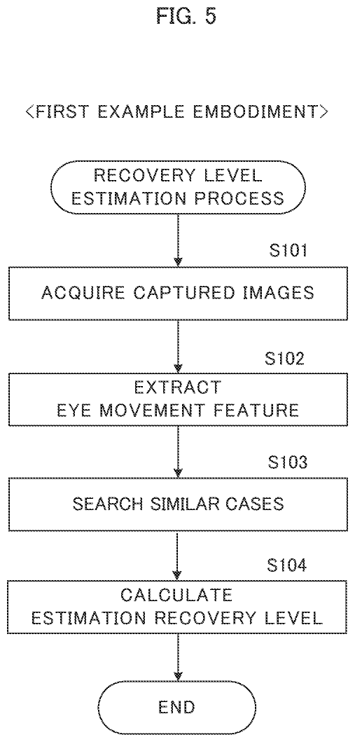

Next, the recovery level estimation process by the recovery level estimation device 1 will be described. FIG. 5 is a flowchart of the recovery level estimation process performed by the recovery level estimation device 1. This recovery level estimation process is realized by the processor 12 depicted in FIG. 2 which executes a program prepared in advance.

First, the recovery level estimation device 1 acquires the captured images D1 obtained by capturing the eyes of the target patient (step S201). Next, the recovery level estimation device 1 extracts the eye movement feature by an image process from the captured images D1 which have been acquired (step S202). Subsequently, the recovery level estimation device 1 searches, among the past cases, for one or more similar cases each including a similar eye movement feature to the eye movement feature of the target patient (step S103). After that, the recovery level estimation device 1 calculates the estimation recovery level of the target patient based on information concerning the recovery levels of the similar cases (step S104). The estimation recovery is presented to the target patient or the medical personnel in any way.

As described above, since the recovery level estimation device 1 estimates the recovery level of the target patient based on the captured images D1 obtained by capturing eyeballs even in a case where the medical personnel or specialist is absent, it is possible to reduce a burden on the medical personnel or the like. Moreover, since a daily recovery level can be predicted even in a sitting position, it is possible for the recovery level estimation device 1 to be applicable for each target patient who has difficulty walking independently without a need for hospital visits or a risk of falling.

Note that the recovery level estimation device 1 stores the calculated estimation recovery level in the memory 13 or the like for each target patient, and outputs an alert to the target patient on the display unit 15 or the like in response to the estimation recovery level of the target patient that is lower than the threshold value.

In addition, the estimation recovery level of the target patient is not limited to one recovery level, and the recovery level estimation device 1 may set the recovery level of all similar cases retrieved as the estimation recovery level of all target patients in a case where the similar cases are searched for a predetermined number of cases from the past cases. In this case, a plurality of estimation recovery levels are presented to the target patient by any method.

As described above, according to the recovery level estimation device 1 of the first example embodiment, it is possible for the target patient to easily and quantitatively measure the estimation recovery level daily at home or elsewhere, and to objectively visualize the daily recovery level. Therefore, it can be expected to increase an amount of rehabilitation due to improved target patient motivation for the rehabilitation, and to improve a quality of rehabilitation through frequent revisions of a rehabilitation plan, thereby improving the effectiveness of recovery. In addition, it is possible to detect an abnormality such as a sign of a recurrent cerebral infarction at an early stage, without waiting for an examination or a consultation by the medical personnel. Examples of industrial applications of the recovery level estimation device 1 include a remote instruction, a management, and the like of the rehabilitation.

### Second Example Embodiment

### Configuration

A recovery level estimation device 1x of a second example embodiment utilizes target patient information concerning a target patient such as an attribute and a recovery record in addition to the eye movement feature, in estimating a recovery level of the target patient. Since a schematic configuration and a hardware configuration of the recovery level estimation device 1x are the same as those of the first example embodiment, the explanations thereof will be omitted.

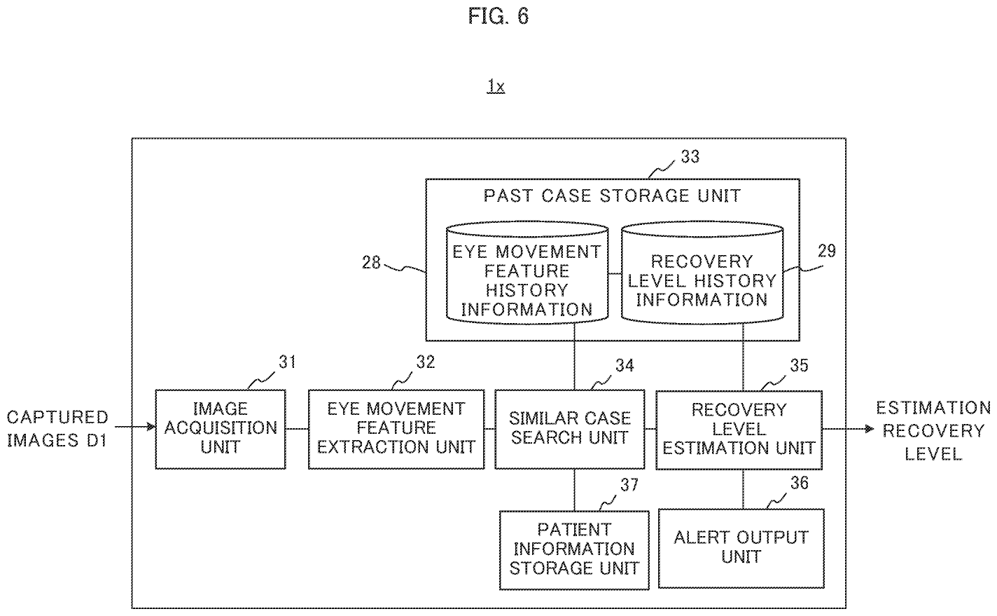

FIG. 6 is a block diagram illustrating a functional configuration of the recovery level estimation device 1x. The recovery level estimation device 1x functionally includes an image acquisition unit 31, an eye movement feature extraction unit 32, a past case storage unit 33, a similar case search unit 34, a recovery level estimation unit 35, an alert output unit 36, a patient information storage unit 37. Note that the image acquisition unit 31, the eye movement feature extraction unit 32, a past case storage unit 33, the similar case search unit 34, the recovery level estimation unit 35, and the alert output unit 36 are realized by the processor 12 executing respective programs. Also, the past case storage unit 33 and the patient information storage unit 37 are realized by the memory 13.

The recovery level estimation device 1x of the second example embodiment searches for one or more similar cases among the past cases by referring to the patient information, calculates the estimation recovery level of the target patient based on recovery levels of the similar cases, and thus estimates the recovery level.

The patient information storage unit 37 stores the patient information concerning each patient having a target disease. The patient information includes a past recovery record of the target patient including information of attributes such as a gender and an age, a history of the recovery level, a disease name, symptoms, rehabilitation contents, and the like, for instance. The patient information storage unit 37 stores the patient information in association with the patient identification information.

The similar case search unit 34 searches for each similar case including a similar eye movement feature to that of the target patient among the past cases by referring to the patient information. By referring to the patient information stored in the patient information storage unit 37, the similar case search unit 34 searches for each similar case in consideration of the attribute and the recovery record of each of the patients included in the past cases and the attribute and the recovery record of the target patient.

Note that since the image acquisition unit 31, the eye movement feature extraction unit 32, the past case storage unit 33, the recovery level estimation unit 35, and the alert output unit 36 are the same as those in the first example embodiment, and the explanations thereof will be omitted.

### Recovery Level Estimation Process

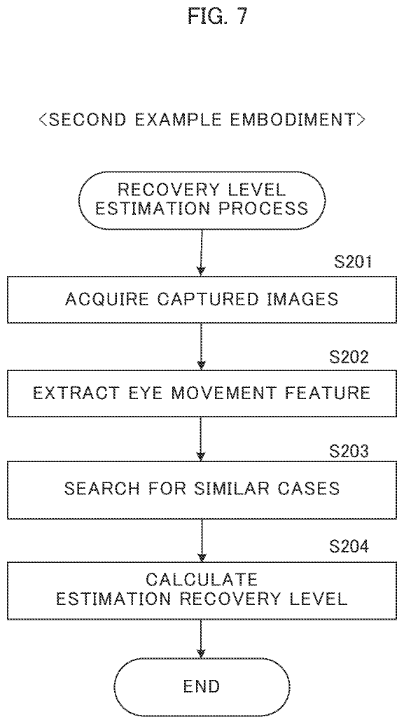

Next, a recovery level estimation process by the recovery level estimation device 1x will be described. FIG. 7 is a flowchart of the recovery level estimation process performed by the recovery level estimation device 1x. This recovery level estimation process is realized by the processor 12 depicted in FIG. 2 which executes a program prepared in advance.

First, the recovery level estimation device 1x acquires the captured images D1 obtained by capturing the eyes of the target patient (step S201). Next, the recovery level estimation device 1x extracts the eye movement feature from the captured images D1 which have been acquired, by an imaging process (step S202). Subsequently, the recovery level estimation device 1x searches for each similar case having eye movement features similar to the eye movement feature of the target patient from among the past cases by referring to the patient information stored in the patient information storage unit 37 (step S203). Next, the recovery level estimation device 1x calculates the estimation recovery level of the target patient based on information concerning the recovery level for each of the similar cases (step S204). After that, the recovery level estimation process is terminated. The estimation recovery level is presented to the target patient, the medical personnel, or the like in any manner.

Note that the recovery level estimation device Ix stores the calculated recovery level in the memory 13 or the like for each target patient, and outputs an alert to the target patient on the display unit 15 or the like when the estimation recovery level of the target patient is lower than the threshold value.

As described above, according to the recovery level estimation device 1x of the second example embodiment, by referring to the patient information, it is possible to retrieve each of the similar cases by considering the attributes and the recovery records of the patients included in the past cases and the attribute and the recovery record of the target patient. That is, it becomes possible for the recovery level estimation device 1x to estimate the recovery level by considering the attribute and the recovery record of each patient.

### Third Example Embodiment

### Configuration

A recovery level estimation device 1y of a third example embodiment presents a task in capturing eyes of a target patient. The task corresponds to a predetermined condition or an assignment related to the eye movement. By presenting the target patient with the task in a case of capturing images of the eyes, the recovery level estimation device 1y is capable of capturing images from which the eye movement feature necessary for estimating the recovery level is easily extracted.

Incidentally, different from the first example embodiment and the second example embodiment, the recovery level estimation device 1y of the third example embodiment internally includes the camera 2. The interface 11, the processor 12, the memory 13, the recording medium 14, the display unit 15, and the input unit 16 are the same as those of the first example embodiment and the second example embodiment, and explanations thereof will be omitted.

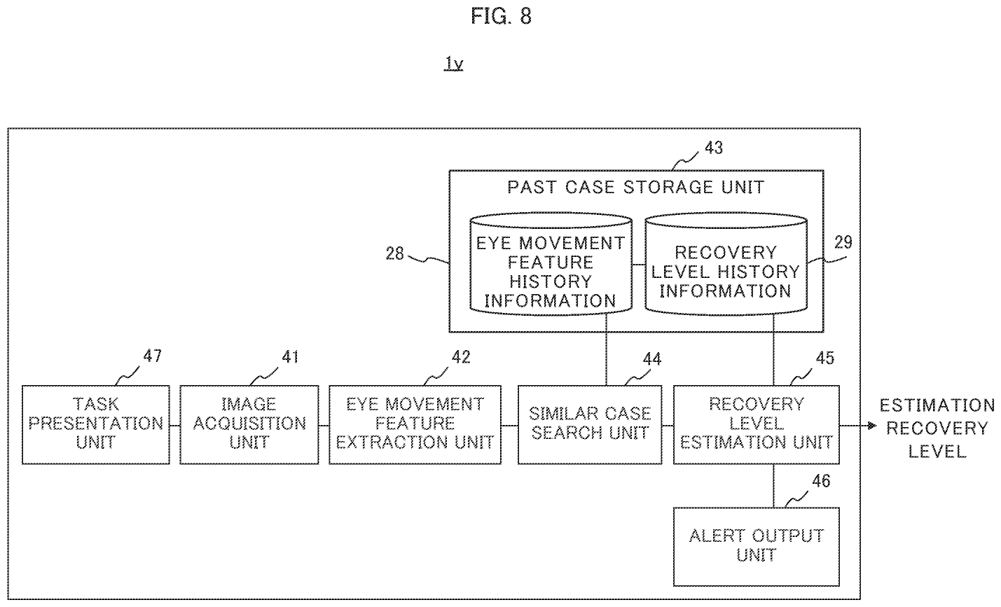

FIG. 8 is a block diagram illustrating a functional configuration of the recovery level estimation device 1y. The recovery level estimation device 1y functionally includes an image acquisition unit 41, an eye movement feature extraction unit 42, a past case storage unit 43, a similar case search unit 44, a recovery level estimation unit 45, an alert output unit 46, and a task presentation unit 47. Note that the image acquisition unit 41, the eye movement feature extraction unit 42, the similar case search unit 44, the recovery level estimation unit 45, the alert output unit 46, and the task presentation unit 47 are realized by the processor 12 executing respective programs. Moreover, the, the past case storage unit 43 is realized by the memory 13.

The recovery level estimation device 1y searches for one or more similar cases among the past cases, and estimates the recovery level by calculating the estimation recovery level of the target patient based on the recovery levels of the retrieved similar cases.

The task presentation unit 47 presents the task to the target patient on the display unit 15. The task is a predetermined condition or an assignment related to the eye movement, and may be arbitrarily set such as “viewing a predetermined image with variation”, “following a moving light spot with the eyes”, or the like, for instance.

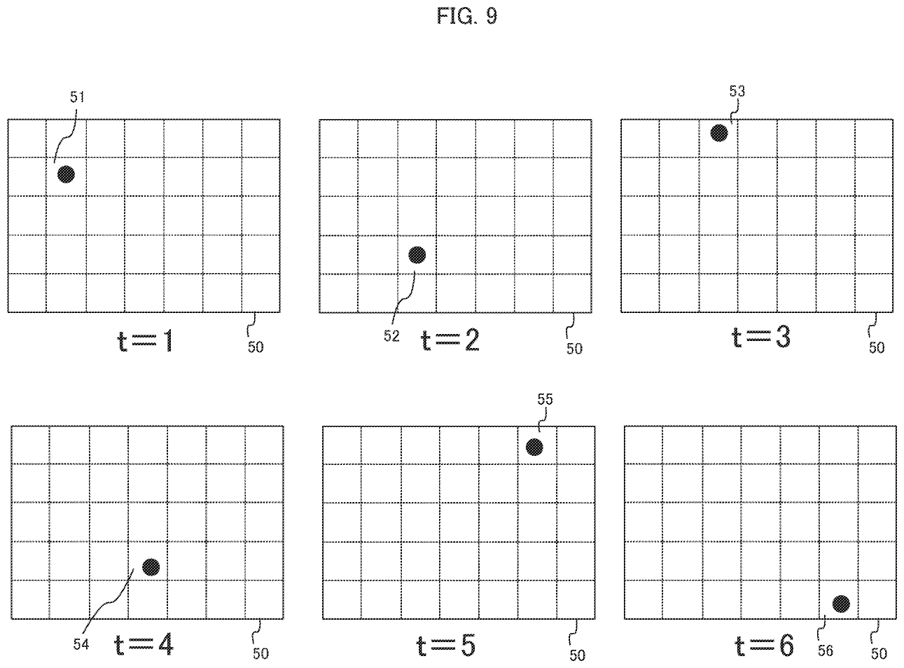

FIG. 9 illustrates a specific example of the task “following a moving light spot with the eyes”. In a light point display region 50 depicted in FIG. 9, a black circle is a light point, and moves to a square 51 at an elapsed time of 1 second (t=1), a square 52 at elapsed time of 2 seconds (t=2), a square 53 at an elapsed time of 3 seconds (t=3), a square 54 at an elapsed time of 4 seconds (t=4), a square 55 at an elapsed time of 5 seconds (t=5), and a square 56 at an elapsed time of 6 seconds (t=6). The target patient tracks the moving light spot over time with the eyes of the target patient. By presenting the task, the camera 2 built into the recovery level estimation device 1y can easily capture images including the visual field defect information of the target patient.

The image acquisition unit 41 acquires the captured images D1 by capturing the eyes moved by the target patient along the task with the camera 2 built into the recovery level estimation device.

Note that since the eye movement feature extraction unit 42, the past case storage unit 43, the similar case search unit 44, the recovery level estimation unit 45. and the alert output unit 46 are the same as those in the first example embodiment, the explanations thereof will be omitted.

### Recovery Level Estimation Process

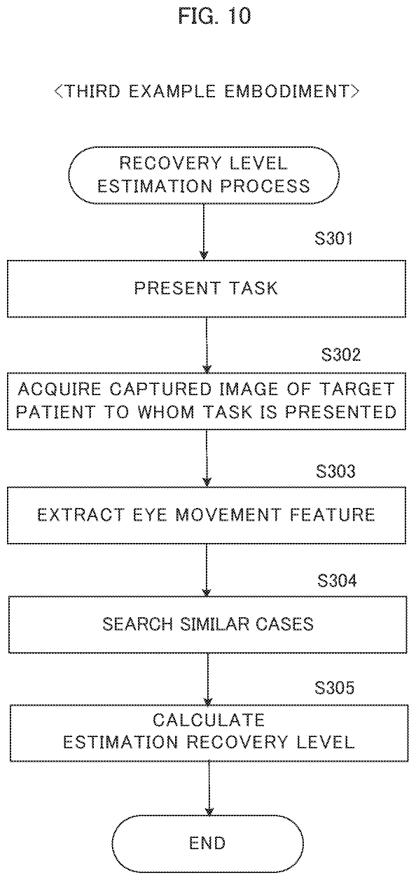

Next, a recovery level estimation process by the recovery level estimation device 1y will be described. FIG. 10 is a flowchart of the recovery level estimation process performed by the recovery level estimation device 1y. This recovery level estimation process is realized by executing a program prepared in advance by the processor 12 depicted in FIG. 2.

First, the recovery level estimation device 1y presents the task to the target patient using the display unit 15 or the like (step S301). Next, the recovery level estimation device 1y captures the eyes of the target patient whom the task is presented, by the camera 2, and acquires the captured images D1 (step S302). In addition, the recovery level estimation device 1y extracts the eye movement feature from the captured images D1 which have been acquired, by the imaging process (step S303). Subsequently, the recovery level estimation device 1y searches for similar cases including eye movement features similar to the eye movement feature of the target patient from among the past cases (step S304). Next, the recovery level estimation device 1y the estimation recovery level of the target patient based on information concerning the recovery levels of the similar cases (step S305). The estimation recovery level is presented to the target patient and the medical personnel in any manner. By presenting a predetermined task as described above, it is possible for the recovery level estimation device 1y to acquire the captured images D1 from which the eye movement feature is easily extracted.

Note that the recovery level estimation device 1y stores the calculated recovery level in the memory 13 or the like for each target patient, and outputs an alert to the target patient to the display unit 15 or the like in response to the estimation recovery level of the target patient which is lower than the threshold value.

Moreover, in the third example embodiment, for convenience of explanations, the recovery level estimation device 1y incorporates the camera 2, and presents the task on the display unit 15. However, the present disclosure is not limited thereto, and the recovery level estimation device does not internally include the camera 2 and is connected to the camera 2 by a wired or wireless communication to exchange data. In this case, the recovery level estimation device 1y outputs the task for the target patient to the camera 2, and acquires the captured images D1 which the camera 2 has been captured.

Moreover, the recovery level estimation device 1y in the third example embodiment may use the patient information described in the second example embodiment. Furthermore, the recovery level estimation device 1 in the first example embodiment and the recovery level estimation device 1x in the second example embodiment may present the task described in this example embodiment.

### Fourth Example Embodiment

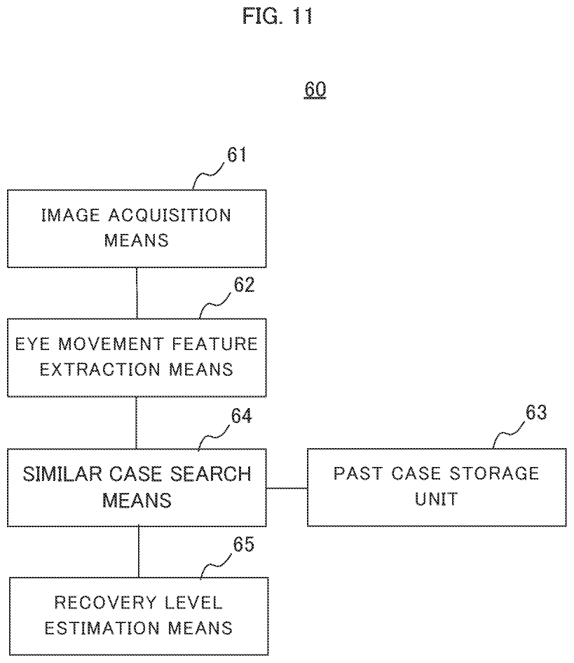

FIG. 11 is a block diagram illustrating a functional configuration of a recovery level estimation device according to a fourth example embodiment. A recovery level estimation device 60 includes an image acquisition means 61, an eye movement feature extraction means 62, a past case storage means 63, a similar case search means 64, and a recovery level estimation means 65.

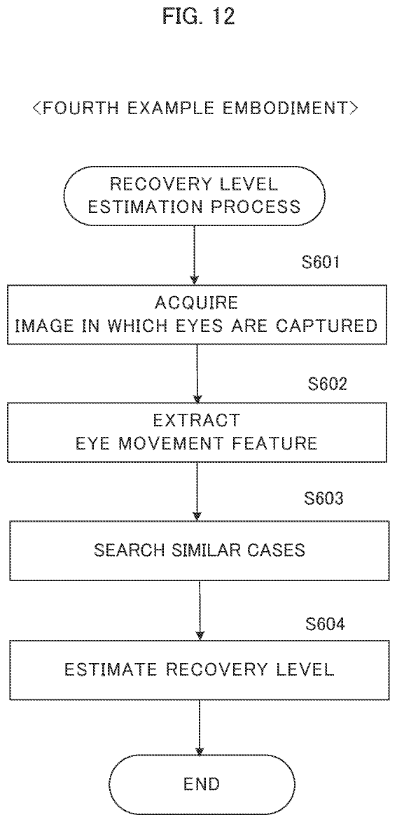

FIG. 12 is a flowchart of a recovery level estimation process performed by the recovery level estimation device 60. The image acquisition means 61 acquires images obtained by capturing the eyes of the target patient (step S601). The eye movement feature extraction means 62 extracts the eye movement feature which is a feature of the eye movement based on the images (step S602). The past case storage means 63 records, as the past cases, a plurality of cases in which each patient and information concerning the eye movement and the recovery level of the patient are associated with to each other. The similar case search means 64 searches for one or more similar cases including eye feature information similar to the eye movement feature of the target patient among the past cases (step S603). The recovery level estimation means 65 estimates the recovery level of the target patient based on the recovery levels of the similar cases (step S604).

According to the recovery level estimation device 60 of the fourth example embodiment, based on the images obtained by capturing the eyes of the target patient, it is possible to estimate the recovery level of the target patient with a predetermined disease.

A part or all of the example embodiments described above may also be described as the following supplementary notes, but not limited thereto.

### Supplementary Note 1

A recovery level estimation device comprising:
an image acquisition means configured to acquire images capturing eyes of a target patient for whom a recovery level is estimated;
an eye movement feature extraction means configured to extract an eye movement feature which is a feature of an eye movement of the target patient, based on the images;
a past case storage means configured to store, as past cases, a plurality of cases in which each of patients and information concerning the eye movement feature and the recovery level of the patient are associated with each other;
a similar case search means configured to search for each similar case including a similar eye movement feature to the eye movement feature of the target patient among the past cases; and
a recovery level estimation means configured to estimate the recovery level of the target patient based on information concerning the recovery level of each similar case.

### Supplementary Note 2

The recovery level estimation device according to supplementary note 1, wherein the eye movement feature includes eye vibration information concerning a vibration of the eyes.

### Supplementary Note 3

The recovery level estimation device according to supplementary note 1 or 2, wherein the eye movement feature includes information concerning any one or more of a bias of a movement direction between the eyes and a misalignment of right and left movements of the eyes.

### Supplementary Note 4

The recovery level estimation device according to any one of supplementary notes 1 to 3, further comprising a task presentation means configured to present a task concerning the eye movement to the target patient, wherein
the image acquisition means acquires the images capturing the eyes of the target patient; and
the eye movement feature extraction means extracts the eye movement feature during the task based on the images.

### Supplementary Note 5

The recovery level estimation device according to supplementary note 4, wherein the eye movement feature includes visual field defect information concerning a visual field defect.

### Supplementary Note 6

The recovery level estimation device according to supplementary note 1, further comprising a patient information storage means configured to store patient information concerning any one or more of an attribute of the patient and a recovery record of the patient for each patient, wherein
the similar case search means searches the similar case among the past cases by referring to the patient information.

### Supplementary Note 7

The recovery level estimation device according to any one of supplementary notes 1 to 6, further comprising an alert output means configured to output an alert upon the recovery level of the target patient that is lower than a threshold value.

### Supplementary Note 8

The recovery level estimation device according to supplementary note 1, wherein
the similar case search means searches for a predetermined number of the similar cases among the past cases, and
the recovery level estimation means estimates the recovery level of the target patient based on a most frequent recovery level in the similar cases.

### Supplementary Note 9

A recovery level estimation method comprising:
acquiring images capturing eyes of a target patient for whom a recovery level is estimated;
extracting an eye movement feature which is a feature of an eye movement of the target patient, based on the images;
searching for each similar case including a similar eye movement feature to the eye movement feature of the target patient among a plurality of past cases in which each of patients and information concerning the eye movement feature and the recovery level of the patient are associated with each other; and
estimating the recovery level of the target patient based on information concerning the recovery level of each similar case.

### Supplementary Note 10

A recording medium storing a program, the program causing a computer to perform a process comprising:
acquiring images capturing eyes of a target patient for whom a recovery level is estimated;
extracting an eye movement feature which is a feature of an eye movement of the target patient, based on the images;
searching for each similar case including a similar eye movement feature to the eye movement feature of the target patient among a plurality of past cases in which each of patients and information concerning the eye movement feature and the recovery level of the patient are associated with each other; and
estimating the recovery level of the target patient based on information concerning the recovery level of each similar case.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

1, 1x, 1y Recovery level estimation device
2 Camera
11 Interface
12 Processor
13 Memory
14 Recording medium
15 Display unit
16 Input unit
21, 31, 41 Image acquisition unit
22, 32, 42 Eye movement feature unit
23, 33, 43 Past case storage unit
24, 34, 44 Similar case search unit
25, 35, 45 Recovery level estimation unit
26, 36, 46 Alert output unit
37 Patient information storage unit
47 Task presentation unit

What is claimed is:

1. A recovery level estimation device comprising:
at least one memory configured to store instructions; and
at least one processor configured to execute the instructions to:
acquire images capturing eyes of a target patient for whom a recovery level is estimated;
extract an eye movement feature which is a feature of an eye movement of the target patient, based on the images;
store, as past cases, a plurality of cases in which each of patients and information concerning the eye movement feature and the recovery level of the patient are associated with each other;
search for each similar case including a similar eye movement feature to the eye movement feature of the target patient among the past cases; and
estimate the recovery level of the target patient based on information concerning the recovery level of each similar case.

2. The recovery level estimation device according to claim 1, wherein the eye movement feature includes eye vibration information concerning a vibration of the eyes.

3. The recovery level estimation device according to claim 1, wherein the eye movement feature includes information concerning any one or more of a bias of a movement direction between the eyes and a misalignment of right and left movements of the eyes.

4. The recovery level estimation device according to claim 1, wherein the processor is further configured to present a task concerning the eye movement to the target patient, wherein
the processor acquires the images capturing the eyes of the target patient; and
the processor extracts the eye movement feature during the task based on the images.

5. The recovery level estimation device according to claim 4, wherein the eye movement feature includes visual field defect information concerning a visual field defect.

6. The recovery level estimation device according to claim 1, wherein the processor is further configured to store patient information concerning any one or more of an attribute of the patient and a recovery record of the patient for each patient, wherein
the processor searches the similar case among the past cases by referring to the patient information.

7. The recovery level estimation device according to claim 1, wherein the processor is further configured to output an alert upon the recovery level of the target patient that is lower than a threshold value.

8. The recovery level estimation device according to claim 1, wherein
the processor searches for a predetermined number of the similar cases among the past cases, and
the processor estimates the recovery level of the target patient based on a most frequent recovery level in the similar cases.

9. A recovery level estimation method comprising:
acquiring images capturing eyes of a target patient for whom a recovery level is estimated;
extracting an eye movement feature which is a feature of an eye movement of the target patient, based on the images;
searching for each similar case including a similar eye movement feature to the eye movement feature of the target patient among a plurality of past cases in which each of patients and information concerning the eye movement feature and the recovery level of the patient are associated with each other; and
estimating the recovery level of the target patient based on information concerning the recovery level of each similar case.

10. A non-transitory computer-readable recording medium storing a program, the program causing a computer to perform a process comprising:
acquiring images capturing eyes of a target patient for whom a recovery level is estimated;
extracting an eye movement feature which is a feature of an eye movement of the target patient, based on the images;
searching for each similar case including a similar eye movement feature to the eye movement feature of the target patient among a plurality of past cases in which each of patients and information concerning the eye movement feature and the recovery level of the patient are associated with each other; and
estimating the recovery level of the target patient based on information concerning the recovery level of each similar case.
